(19) United States
(12) Patent Application Publication
(10) Pub. No.: US 2009/0075374 A1
(43) Pub. Date: Mar. 19, 2009

(54) METHODS OF GENERATING EPITHELIAL LINEAGE CELLS FROM EMBRYOID BODIES AND PLURIPOTENT CELLS

(76) Inventors: Sean P. Palecek, Verona, WI (US); Juan J. De Pablo, Madison, WI (US); Lin Ji, Arlington, MA (US); Christian M. Metallo, Cambridge, MA (US)

Correspondence Address: QUARLES & BRADY LLP, 33 E. MAIN ST, SUITE 900, P.O. BOX 2113, MADISON, WI, 53701-2113, US

(21) Appl. No.: 12/179,462

(22) Filed: Jul. 24, 2008

Related U.S. Application Data
(60) Provisional application No. 60/951,530, filed on Jul. 24, 2007.

(51) Int. Cl. C12N 5/08

(52) U.S. Cl. 435/366; 435/375

(57) ABSTRACT

Methods of generating p63-positive cells from embryoid bodies and pluripotent cells by culturing the cells in the presence of a retinoid and optionally a bone morphogenetic protein, such that the cells express at least p63. The p63-positive cells can be further cultured without the retinoid and optional bone morphogenetic protein to K14-positive cells. The K14-positive cells can be further cultured into various terminally differentiated cell types of the epithelial lineage.

### CROSS-REFERENCE TO RELATED APPLICATIONS

This application claims the benefit of U.S. Provisional Patent Application No. 60/951,530, filed Jul. 24, 2007, incorporated herein by reference as if set forth in its entirety.

### STATEMENT REGARDING FEDERALLY SPONSORED RESEARCH OR DEVELOPMENT

This invention was made with United States government support awarded by the following agency: NSF 0520527. The United States government has certain rights in this invention.

### BACKGROUND

The invention relates generally to methods of generating epithelial precursors (p63⁺ cells) and epithelial lineage cells (K14⁺), and relates more particularly to methods using retinoids and bone morphogenetic proteins to generate p63⁺ cells, which can then be further cultured into epithelial cells.

Human embryonic stem cells (hESCs) are pluripotent cells having an extensive proliferative capacity and an ability to differentiate into one of three embryonic germ layers. However, to exploit the expansion potential of undifferentiated pluripotent cells, including hESCs, efficient differentiation processes must be developed to generate and culture lineage-restricted progenitors at high purity, while eliminating pluripotent cells and other non-desired differentiated cell types from the culture.

Of particular interest herein are epithelial precursor cells generated from pluripotent cells, particularly hESCs. Hallmarks of an epithelial precursor include expression of p63, a transcription factor for maintaining regenerative epithelia (i.e., the epidermis) and in some cases cytokeratin 18 (K18).

Retinoids such as retinoic acid (RA) are known regulators of cell proliferation and differentiation that are involved in embryonic development. Bamberger et al. showed that RA inhibited terminal differentiation of one type of epithelial cells, keratinocytes, in vitro by modulating p63 expression. Bamberger C, et al., “Retinoic acid inhibits downregulation of DeltaNp63alpha expression during terminal differentiation of human primary keratinocytes,” J. Invest. Dermatol. 118:133-138 (2002). On the other hand, RA also directs ESC-derived neuroepithelia to become motor neurons. See Wichterle H, et al., “Directed differentiation of embryonic stem cells into motor neurons,” Cell 110:385-397 (2002).

Recently, Coraux et al. showed that bone morphogenetic protein-4 (BMP-4) induced epidermal differentiation in murine ESCs (mESCs). Coraux C, et al., “Reconstituted skin from murine embryonic stem cells,” Curr. Biol. 13:849-853 (2003). While BMP-4 signaling plays a role in early ectodermal fate during early development in other species, its role in human ectodermal development is unknown. BMP-4, however, can direct hESCs to non-ectodermal lineages.

Although pluripotent cells, such as ESCs, can differentiate into epithelial cells, current methods are inefficient, and the isolated cells have a reduced expansion potential. In addition, the art is limited by an inability to isolate a sufficient number of epithelial precursors for subsequent differentiation and use. For example, Green et al. generated keratinocytes through an embryoid body (EB) intermediate, but the keratinocytes lacked a growth potential commensurate with post-natal keratinocytes. Green H, et al., “Marker succession during the development of keratinocytes from cultured human embryonic stem cells,” Proc. Natl. Acad. Sci. USA 100: 15625-15630 (2003). Like Green et al., Tuchi et al. generated keratinocytes from hESCs through an EB intermediate, but the cells had a low proliferative potential in culture and did not expand in mass culture. Iuchi S, et al., “Immortalized keratinocyte lines derived from human embryonic stem cells,” Proc. Natl. Acad. Sci. USA 103:1792-1797 (2006). Thus, it remains difficult to obtain epithelial cells suitable for clinical applications.

For the foregoing reasons, there remains a need for epithelial precursors generated from ESCs for use in research and clinical applications.

### BRIEF SUMMARY

In a first aspect, the present invention is summarized as a method of generating p63-positive cells that includes the step of culturing embryoid bodies (EBs) in a defined differentiation medium containing a sufficient amount of a retinoid, and optionally also containing a sufficient amount of a bone morphogenetic protein (BMP), for at least about two days, but alternatively from about four days to about nine days, to obtain an essentially pure population of p63-positive cells. As used herein, “about” means within 5% of a stated concentration range or within 5% of a stated time frame; “essentially pure” means that greater than at least about 85%, but preferably at least about 95%, of the cells express a stated marker, such as p63, as determined by flow cytometry; and “a sufficient amount” means a concentration of the retinoid and/or BMP that produces a cell population in which at least greater than 85% of the cells are p63⁺.

In a second aspect, the present invention is summarized as another method of generating p63-positive cells that includes the step of culturing pluripotent cells in a defined differentiation medium containing a sufficient amount of a retinoid, and optionally also containing a sufficient amount of a BMP, for at least about four days, but alternatively from about six days to about seven days, to obtain an essentially pure population of p63-positive cells.

In some embodiments of either aspect, the EBs or pluripotent cells are of human origin; however, when the cells are pluripotent cells, they can be ESCs or induced pluripotent stem cells.

In some embodiments of either aspect, the retinoid is RA and the BMP is BMP-4.

In some embodiments of either aspect, the population of p63-positive cells are also K18⁺.

In a third aspect, the present invention is summarized as a method of generating K14-positive cells that includes the step of plating the p63-positive cells onto an adherent surface, such as a matrix-coated dish, in a defined serum-free culture medium without a retinoid or BMP to obtain an essentially pure population of K14-positive cells, which may continue to express p63.

In a fourth aspect, the present invention is summarized as a method of generating filaggrin- and involucrin-positive cells that includes the step of plating K14-positive cells onto an adherent surface in calcium (Ca²⁺)-containing medium to obtain an essentially pure population of filaggrin- and involucrin-positive cells, which may additionally express K10.

In a fifth aspect, the present invention is summarized as a method of generating K3/K12-positive cells that includes the step of plating K14-positive cells onto an adherent surface in a calcium-free medium to obtain K3/K12-positive cells.

In a sixth aspect, the invention is summarized as an essentially pure population of K14+ cells produced from the methods described above.

In a seventh aspect, the invention is summarized as an essentially pure population of filaggrin- and involucrin-positive cells.

These and other features, aspects and advantages of the present invention will become better understood from the description that follows. The description of preferred embodiments is not intended to limit the invention to cover all modifications, equivalents and alternatives. Reference should therefore be made to the claims herein for interpreting the scope of the invention.

### DESCRIPTION OF PREFERRED EMBODIMENTS

The present invention relates to the inventors' observation that retinoids, such as RA, applied to EBs or pluripotent cells mediate epithelial differentiation in conjunction with BMP signaling. This observation suggests that retinoids can be used to obtain essentially pure populations of EB- or pluripotent cell-derived epithelial precursor cells that can subsequently form terminally differentiated coherent epithelial sheets. See, Metallo C, et al., “Retinoic acid and bone morphogenetic protein signaling synergize to efficiently direct epithelial differentiation of human embryonic stem cells,” Stem Cells 26:372-380 (2008), incorporated herein by reference as if set forth in its entirety.

When induced at an early stage with retinoids, essentially pure epithelial cell populations obtained from EBs or pluripotent cells expressed terminal differentiation markers and formed coherent epithelial sheets. This differentiation process, coupled with a self-renewal capacity of undifferentiated pluripotent cells, provided a means of generating large quantities of non-transformed epithelial cells.

Whereas residual Oct4⁺, undifferentiated pluripotent cells may be present in cultures after previous differentiation methods, the present methods employing retinoids advantageously leave no detectable Oct4⁺ undifferentiated cells that could give rise to teratomas if present in clinical cell preparations. As such, the methods described herein ultimately generated essentially pure populations of p63-positive and K14-positive cells that may be suited for clinical use.

The present invention therefore broadly relates to novel methods for differentiating EBs and pluripotent cells into p63-positive cells by providing a medium having a retinoid and optionally a BMP during differentiation. The p63-positive cells can then become K14⁺ when cultured on an adherent surface in a medium lacking the retinoid and optional BMP. The inventors contemplate that the K14-positive cells can be further differentiated into any of the various cells of the epithelial lineage including, but not limited to, keratinocytes, corneal epithelial cells, bulge keratinocytes of the hair follicle, intestinal epithelial cells, bronchial epithelial cells, bladder epithelial cells, olfactory epithelial cells, mammary epithelial cells and prostate epithelial cells.

In the methods described herein, one can use EBs as the starting cell type. As used herein, “embryoid bodies” or “EBs” mean an aggregate of cells derived from pluripotent cells, such as ESCs or induced pluripotent stem (iPS) cells, where cell aggregation can be initiated by hanging drop, by plating upon non-tissue culture treated plates or by spinner flasks (i.e., low-attachment conditions), as well as by any method that prevents the cells from adhering to a surface to form typical colony growth. EBs appear as rounded collections of cells and contain cell types derived from all three germ layers (i.e., the ectoderm, mesoderm and endoderm). Methods for generating EBs are well known to one of ordinary skill in the art. See, e.g., Itskovitz-Eldor J, et al., “Differentiation of human embryonic stem cells into embryoid bodies compromising the three embryonic germ layers,” Mol. Med. 6:88-95 (2000); Odorico J, et al., “Multilineage differentiation from human embryonic stem cell lines,” Stem Cells 19:193-204 (2001); and U.S. Pat. No. 6,602,711, each of which is incorporated herein by reference as if set forth in its entirety.

Preferably, one uses pluripotent cells as the starting cell type. As used herein, “pluripotent cells” mean cells that can differentiate into all three germ layers (e.g., endoderm, mesoderm and ectoderm). Pluripotent cells express a variety of pluripotent cell-specific markers, have a cell morphology characteristic of undifferentiated cells (i.e., compact colony, high nucleus to cytoplasm ratio and prominent nucleolus) and form teratomas when introduced into an immunocompromised animal, such as a SCID mouse. See, e.g., Evans M & Kaufman M, “Establishment in culture of pluripotential cells from mouse embryos,” Nature 292:154-156 (1981). The teratomas typically contain cells or tissues characteristic of all three germ layers. One of ordinary skill in the art can assess these characteristics by using techniques commonly used in the art. See, e.g., Thomson J, et al., “Embryonic stem cell lines derived from human blastocysts,” Science 282:1145-1147 (1998), incorporated herein by reference as if set forth in its entirety.

Pluripotent cells are capable of both proliferation in cell culture and differentiation towards a variety of lineage-restricted cell populations that exhibit multipotent properties. Multipotent somatic cells are more differentiated relative to pluripotent cells, but are not terminally differentiated. Pluripotent cells therefore have a higher potency than multipotent cells.

Suitable pluripotent cells include, but are not limited to, ESCs or induced pluripotent stem (iPS) cells, and preferably are from a primate, especially a human primate. As used herein, “embryonic stem cells” or “ESCs” mean pluripotent cells derived from an inner cell mass of a blastocyst. See, Thomson et al., supra. These cells express at least Oct-4, SSEA-3, SSEA-4, TRA-1-60 or TRA-1-81, and appear as compact colonies having a high nucleus to cytoplasm ratio and prominent nucleolus. ESCs are commercially available from sources such as WiCell Research Institute (Madison, Wis.). When using ESCs, at least 90% of the starting population should be undifferentiated.

As used herein, “induced pluripotent stem cells” or “iPS cells” mean pluripotent cells that are substantially genetically identical to their respective differentiated somatic cell of origin and display characteristics similar to higher potency cells, such as ESCs. iPS cells can be obtained from various differentiated (i.e., non-pluripotent and multipotent) somatic cells. These cells are reprogrammed differentiated somatic cells that may vary with respect to their differentiated somatic cell of origin, that may vary with respect a specific set of potency-determining factors and that may vary with respect to culture conditions used to isolate them, but nonetheless are substantially genetically identical to their respective differentiated somatic cell of origin and display characteristics similar to higher potency cells (e.g., express at least Oct-4, SSEA-3, SSEA-4, TRA-1-60 or TRA-1-81, but not SSEA-1; appear as compact colonies having a high nucleus to cytoplasm ratio and prominent nucleolus; may express alkaline phosphatase (ALP); and differentiate into cells characteristic of all three germ layers) such as ESCs. See, e.g., Yu J, et al., “Induced pluripotent stem cell lines derived from human somatic cells,” Science 318:1917-1920 (2007), incorporated herein by reference as if set forth in its entirety.

Other types of pluripotent cells include, but are not limited to, cells from somatic cell nuclear transfer (see, Wilmut I, et al., “Viable offspring derived from fetal and adult mammalian cells,” Nature 385:810-813 (1997)) or cells from fusion of somatic cells with ESCs (see, Cowan C, et al., “Nuclear reprogramming of somatic cells after fusion with human embryonic stem cells,” Science 309:1369-1373 (2005); and Yu et al., “Human embryonic stem cells reprogram myeloid precursors following cell-cell fusion,” Stem Cells 24:168-176 (2006)).

Suitable retinoids can be any naturally occurring or synthetic retinoid. As used herein, a “retinoid” means a class of chemical compounds related chemically to vitamin A and having at least a cyclic end group, a polyene side chain and a polar end group. Although tretinoin (all-trans RA) was used below, the present invention is not intended to be limited to this retinoid. Other suitable retinoids include, but are not limited to, first generation retinoids, such as retinol (Vitamin A), retinal (Vitamin A aldehyde), alitretinoin (9-cis RA), neotretinoin (11-cis-RA) and isotretinoin (13-cis RA); second generation retinoids, such as etretinate and its metabolite acitretin; and third generation retinoids, such as tazarotene and bexarotene. Other suitable retinoids can be 9,11-di-cis retinol, and 11,13-di-cis retinol, as well as physiologically compatible ethers, esters, amides and salts thereof. Preferably, the retinoid is tretinoin, alitretinoin or isotretinoin. It is also contemplated that small molecule agents that activate the RAR or RXR receptor can be used, such as a RAR agonist, such as CD336 (Galderma; Sophia Antipolis, France). The retinoid concentration can vary from about 10⁻⁸ M to about 10⁻⁵ M, from about 0.1 μM to less than about 10 μM, from about 1.0 μM to about 5.0 μM, but also can be about 1.0 μM. The cells can be exposed to the retinoid for about two days to about nine days, and preferably for about seven days. One of ordinary skill in the art understands that retinoids easily degrade, especially in light; therefore, the actual retinoid concentration may vary with prolonged culture. Thus, and as noted below, the inventors found that medium changes are preferably about every other day. Once formed, EBs can be immediately exposed to the retinoid in culture. However, when the cells are pluripotent cells, they can be exposed to the retinoid starting about twelve hours after plating, but preferably about twenty-four hours after plating (i.e., once the cells become attached).

Suitable BMPs can be any BMP. As used herein, “bone morphogenetic protein” or “BMP” means a protein belonging to a group of growth factors and cytokines known for their ability to induce the formation of bone and cartilage, especially those belonging to the transforming growth factor beta (TGF-β) superfamily of proteins (i.e., BMP-2, BMP-3, BMP-4, BMP-5, BMP6 and BMP-7). Although BMP-4 was used below, the present invention is not intended to be limited to this particular BMP. Other suitable BMPs include, but are not limited to, BMP-2 and BMP-7. Alternatively, the BMP can be any agent that interacts with the BMP-4 receptor, such as a BMP-4 analog, or any agent that activates the same downstream elements as BMP-4, such as agents that induce sMAD 1/5/8 phosphorylation. The BMP concentration can vary from about 5 ng/ml to about 50 ng/ml, but also can be about 25 ng/ml. The cells can be exposed to the BMP for about two days to about nine days, but also can be exposed for about seven days. Once formed, EBs can be immediately exposed to the BMP in culture. However, when the cells are pluripotent cells, they can be exposed to the BMP starting about twelve hours after plating, but can be exposed about twenty-four hours after plating (i.e., once the cells become attached).

When differentiating EBs or pluripotent cells into p63-positive cells, the cells can be cultured in any medium used to differentiate cells into epithelial precursors. For example, one can culture the cells in an unconditioned medium, conditioned medium, chemically defined medium (e.g., TeSR™ and X-Vivo) or N2 medium, along with a retinoid and BMP as indicated above. Another characteristic of the medium is that it should also maintain PI3K activation (i.e., contains insulin). Regardless of the medium used, medium changes can be at least every other day.

Preferably, the cells are differentiated in a chemically defined medium along with a retinoid and BMP as indicated above. As used herein, “chemically defined,” “defined culture medium” or “defined medium” means that the medium has known quantities of all ingredients and is serum-free. As used herein, “serum-free” means that neither the culture nor the culture medium contains serum or plasma, although purified or synthetic components of serum or plasma (e.g., FGFs) can be provided in the culture in reproducible amounts. As such, serum that is normally added to culture medium for cell culture is replaced by known quantities of serum components, such as, e.g., albumin, insulin, transferrin and possibly specific growth factors (i.e., bFGF, TGF or PDGF). Likewise, the medium is free of feeder cells, conditioned medium and mouse embryonic feeder (MEF) cells. Defined medium includes, but is not limited to, TeSR™ or mTeSR™, which are commercially available from StemCell Technologies (Vancouver, Canada). See also, Ludwig T, et al, “Derivation of human embryonic stem cells in defined conditions,” Nat. Biotechnol. 24:185-187 (2006); and Ludwig T, et al., “Feeder-independent culture of human embryonic stem cells,” Nat. Methods 3:637-646 (2006), each of which is incorporated herein by reference as if set forth in its entirety.

During culture, conventional cell culture conditions can be used. For example, the temperature can vary between about 36° C. to about 37.5° C. Likewise, the CO₂ concentration can, and will, vary between about 2% to about 10% depending on the medium and bicarbonate concentration. For example, the cell culture conditions can be 37° C. and 5% CO₂ in a humidified chamber.

When differentiating the p63-positive cells into K14-positive cells, the p63-positive cells can be cultured in any epithelial (i.e., keratinocyte) medium. For example, the cells can be cultured in a defined keratinocyte medium that is serum-free for about two days to about nine days. Defined keratinocyte medium is well known in the art and is commercially available from sources such as 3H Biomedical AB (Uppsala, Sweden), Invitrogen (Carlsbad, Calif.), Millipore (Billerica, Mass.), Lonza (Walkersville, Md.) and ScienCell Research Laboratories (Carlsbad, Calif.). Unlike the defined culture medium, the differentiation medium does not contain the retinoid or BMP. The medium can be changed every third day, but is preferably changed at least every other day.

During differentiation to K14-positive cells, the cells can be cultured on an adherent surface. For example, the cells can be cultured on a basement membrane matrix surface. Suitable matrix materials include, but are not limited to, collagen (e.g., Collagen type I and/or Collagen type IV), fibronectin, gelatin, glycosaminoglycans, laminin, Matrigel® osteocalcin and osteonectin, which all may be suitable as an extracellular matrix by themselves or in various combinations.

During differentiation, conventional cell culture conditions and medium changes can be used, as described above. Typically, however, the differentiating cells can be passaged after three to four weeks, depending on the initial plating density.

After differentiation, the resulting K14-positive cells display consistent epithelial morphology (i.e., tightly packed with a cobblestone-like morphology at high density; cells will spread out more at lower density and the morphology is less uniform), express K14, p63 (in some instances) and E-cadherin, but lack Brachyury (T), FOXA2, Oct4 and Sox1 expression. At least 85% of the resulting cells express K14; typically, however, at least about 95% of the resulting cells express K14.

As indicated above, the K14-positive cells can be further differentiated to express terminal differentiation markers characteristic of specific cells types in the epithelial lineage. For example, one can obtain keratinocytes by culturing K14-positive cells on an adherent surface, such as Collagen IV, in a defined serum-free medium in the presence of calcium (i.e., about 0.5 mM to about 2 mM, or about 1 mM) to confluence (i.e., about 8 days). The specific cell density of the starting culture is not important (i.e., about 2,500 cells/cm² to about 25,000 cells/cm², or about 2,500 cells/cm² to about 50,000 cells/cm², or about 20,000 cells/cm²). With lower plating densities, the cells take longer to reach confluence. The keratinocytes can form intact epithelial sheets (i.e., stratified keratinocytes) when detached from the surface (i.e., by any of the chemical, mechanical or enzymatic treatment known to one or ordinary skill in the art, such as, e.g., by Dispase® treatment) and are characterized as further expressing filaggrin, involucrin and optionally K10. Alternatively, one can obtain corneal epithelial cells by culturing K14-positive cells on an adherent surface, such as Collagen IV, in a defined serum-free medium (no calcium in the medium) to confluence (i.e., about 8 days). The corneal epithelial cells are characterized as further expressing K3/K12.

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are described herein.

The invention will be more fully understood upon consideration of the following non-limiting Examples.

### EXAMPLES

### Example 1

### RA and BMP-4 Signaling Synergize to Efficiently Direct Epithelial Differentiation of hESCs

Methods: H1 and H9 hESC lines (WiCell Research Institute) were maintained on a layer of mouse embryonic fibroblasts (MEFs) in unconditioned medium (UM) containing DMEM/F12 containing 20% Knockout Serum Replacer, 1×MEM non-essential amino acids (Sigma; St. Louis, Mo.), 1 mM L-glutamine, 0.1 mM β-mercaptoethanol and 4 mg/ml bFGF. Alternatively, hESCs were plated on Matrigel® in MEF-conditioned medium (MEF-CM) and passaged every five to six days using Dispase®.

In some experiments, K14-positive cells were generated from EBs. EBs were formed via enzymatic detachment of hESC colonies and cultured either in UM without bFGF or in N2 medium comprising: DMEM/F12 containing 1×N2 supplement and MEM non-essential amino acids. After one to two days, EBs were transferred to a new vessel to remove adherent MEFs and allowed to differentiate in either N2 medium or UM supplemented with 1 μM all-trans RA and 25 ng/ml BMP-4 (R&D Systems; Minneapolis, Minn.) and/or 125 ng/ml Noggin (R&D Systems), with medium changes every other day. Differentiated EBs were then plated onto gelatin-coated plates in Defined Keratinocyte Serum-Free Medium (DSFM; Invitrogen) without RA and BMP-4, with medium changes every other day.

Alternatively, K14-positive cells were generated by direct differentiation of hESCs. hESC colonies were grown for five to seven days on Matrigel® in MEF-CM before switching to a differentiation medium containing a combination of the following: DMSO, 1 μM all-trans RA, 25 ng/ml BMP-4 and/or 125 ng/ml Noggin. After six to seven days, differentiated cells were removed with Dispase® and optionally cultured in a suspension overnight before plating onto gelatin- or collagen-coated plates in DSFM without RA and BMP-4, with medium changes every other day.

Primary human keratinocytes (Invitrogen) and hESC-derived K14-positive cells were sub-cultured on Collagen IV- (Sigma; St. Louis, Mo.) coated plates in DSFM. All medium and additives were from Invitrogen unless otherwise noted.

For immunohistochemistry, cells were fixed in 4% paraformaldehyde for 15 minutes at room temperature before blocking and permeabilizing with 5% milk in PBS with 0.4% Triton X-100. Primary antibodies were incubated overnight at 4° C. in blocking buffer and either mouse anti-p63, mouse anti-K14, rabbit anti-K14, mouse anti-K10 (all from Lab Vision; Fremont, Calif.), mouse anti-K3/K12 (Chemicon; Temecula, Calif.), mouse anti-Oct4, mouse anti-nestin, goat anti-filaggrin or goat anti-involucrin (Santa Cruz Biotechnology; Santa Cruz, Calif.). Cells were stained with an appropriate fluorophore-conjugated secondary antibody (Invitrogen) for one hour at room temperature and Hoechst dye. Immunofluorescence images were observed on an Olympus IX70 microscope (Leeds Precision Instruments, Minneapolis, Minn.) using MetaVue™ imaging software (MDS Analytical Technologies; Downingtown, Pa.).

For flow cytometry, cells were detached from culture plates using Trypsin-EDTA and 2% chick serum, fixed in 1% paraformaldehyde for 10 minutes at 37° C. and permeabilized on ice in 90% methanol. Primary antibodies (described above) were incubated overnight in PBS with 2% fetal calf serum (FCS) and 0.1% NaN₃ at 1:100. Control samples were included using isotype-specific or no primary antibody. After one hour incubation with secondary stain, cells were analyzed on a FACScalibur™ flow cytometer using CellQuest™ software (both from BD Biosciences; San Diego, Calif.).

For semi-quantitative and quantitative PCR, RNA was harvested from cells using a RNeasy® Mini kit (Qiagen; Valencia, Calif.). cDNA was then generated using Omniscript® RT (Qiagen), 1 μg RNA and oligo-dT primers. For semi-quantitative analysis, PCR was conducted using Platinum® Taq (Invitrogen) with 1 μl cDNA for 30 or 35 cycles. For quantitative analysis, PCR was conducted using Quantitect® SYBR Green qPCR kit (Qiagen) with 1 μl cDNA on an iCycler (BioRad; Hercules, Calif.). Relative expression levels were calculated using the ΔCT method, normalizing to GAPDH transcription. Primers (Table 1) were designed to span intron or bound exon borders, and PCR products were verified by melt curve analysis and/or 2% agarose gel electrophoresis.

TABLE 1
Primers for assessing epithelial lineage differentiation.

| | | | |
|---|---|---|---|
| | | | Amplicon |
| Gene | Forward primer | Reverse primer | size |

| | | | |
|---|---|---|---|
| GAPDH | caccgtcaaggctgagaacg | gccccacttgattttggagg | 91 |
| | SEQ ID NO:1 | SEQ ID NO:2 | |
| FOXA2 | gggagcggtgaagatgga | tcatgttgctcacggaggagta | 89 |
| | SEQ ID NO:3 | SEQ ID NO:4 | |
| Brachyury | tgcttccctgagacccagtt | gatcacttctttcctttgcatcaag | 121 |
| | SEQ ID NO:5 | SEQ ID NO:6 | |
| Nestin | tgaagggcaatcacaacagg | tgaccccaacatgacctctg | 136 |
| | SEQ ID NO:7 | SEQ ID NO:8 | |
| Sox1 | caatgcggggaggagaagtc | ctctggaccaaactgtggcg | 464 |
| | SEQ ID NO:9 | SEQ ID NO:10 | |
| Pax6 | ggcaggtattacgagactgg | Cctcatctgaatcttctccg | 427 |
| | SEQ ID NO:11 | SEQ ID NO:12 | |
| K18 | ccgtcttgctgctgatgact | ggccttttacttcctcttcgtg | 200 |
| | SEQ ID NO:13 | SEQ ID NO:14 | |
| TAp63 | aagatggtgcgacaaacaag | agagagcatcgaaggtggag | 234 |
| | SEQ ID NO:15 | SEQ ID NO:16 | |
| ΔNp63 | ggaaaacaatgcccagactc | gtggaatacgtccaggtggc | 294 |
| | SEQ ID NO:17 | SEQ ID NO:18 | |
| K14 | gaccattgaggacctgagga | attgatgtcggcttccacac | 157 |
| | SEQ ID NO:19 | SEQ ID NO:20 | |

For Western blot analysis, cellular protein was harvested using RIPA buffer (Santa Cruz Biotechnology) and quantified using a BCA assay (Pierce; Rockford, Ill.). Equal amounts of protein were resolved on a 10% polyacrylamide gel and transferred to a nitrocellulose membrane. After blocking with 5% milk in PBS, membranes were probed with primary antibodies (described above) overnight and stained with HRP-conjugated antibodies for 1 hour. Protein levels were detected by chemiluminescence (Pierce), and protein loading was verified by probing against β-actin.

For a colony-forming assay, hESC-derived K14-positive cells were trypsinized and plated on Collagen I-coated, six-well plates (BD Biosciences) in DSFM at a density of 5,000 cells/well. Cells were cultured with medium changes every other day for 2 weeks before being fixed with 4% paraformaldehyde and stained with 0.5% rhodamine B for 30 minutes. After a brief wash, the plates were dried, and colonies were counted in triplicate wells.

Results: RA induced epithelial gene expression in EBs, which readily differentiated into epithelial precursors. RA induced newly-formed EBs to form translucent, spherical outgrowths; whereas control EBs retained a more uniform morphology. Quantitative PCR showed a pronounced decrease in Oct-4 expression in the presence of RA. In addition, no mesoderm or endoderm markers (i.e., Brachyury and FoxA2) were detected, indicating that RA-treated EBs were likely differentiating into ectodermal cells. The first markers specific to the neuroectoderm expressed in the differentiating hESCs are Nestin, Pax6 and Sox1; however, transcription of these genes was attenuated or undetectable in RA-treated EBs. p63 was significantly upregulated in RA-treated EBs. In addition, both trans-activating (TA) and dominant negative (ΔN) p63 isoforms were detected in RA-treated EBs, with ΔNp63 expressed at the highest levels. Upon exposure to gelatin-coated plates and culture in keratinocyte medium lacking RA, virtually all cells previously treated with RA expressed p63, while control cultures showed minimal p63 expression. Some cells also expressed another epithelial marker, K18.

Culture of adherent RA-treated EBs in keratinocyte medium generated significant populations of K14⁺ cells, as determined by flow cytometry. The percentage of K14⁺ cells observed in RA-treated EBs increased for twenty-five days after plating and was significantly greater than untreated EBs. Upon adhesion to gelatin, RA-treated EBs formed monolayer colonies lacking undifferentiated (i.e., Oct-4 expressing) hESCs; whereas untreated EBs attached as large cellular aggregates, often containing undifferentiated hESCs expressing Oct-4. Immunocytochemistry demonstrated that nearly all RA-treated colonies contained K14⁺/p63⁺ cells proliferating and migrating at the periphery. However, further analysis identified some colonies as Nestin⁺, indicating that neuroepithelial cells were a common contaminant in both RA cultures and controls.

RA-treated EBs also showed concentration- and temporal-dependent differentiation effects. To study the concentration-dependent effect of RA, EBs were differentiated with varying RA concentrations for nine days and subsequently cultured on gelatin in DSFM for nineteen days. Significant epithelial lineage induction occurred in RA-treated EBs cultured with about 1 μM all-trans RA. Higher concentrations (e.g., 10 μM) of RA appeared toxic to undifferentiated hESCs and decreased Nestin⁺ populations. To study the temporal-dependent effect of RA, RA was added to the cultures at various stages of differentiation (e.g., either during EB induction or during adherent culture in DSFM). RA exposure for four to eight days during EB induction, but not after plating on gelatin, increased K14⁺ cells. In contrast, RA exposure during adherent culture decreased or eliminated epithelial differentiation, depending upon whether the EBs were exposed to RA during EB induction. When exposed to RA, plated cells from control EBs were enriched in neural precursor populations, as confirmed by qPCR analysis. RA therefore exhibited differential effects on ectodermal fate depending on when the cells were exposed to it.

Likewise, RA induced epithelial gene expression in hESCs during the direct differentiation protocol. After six to seven days of culture in the presence of RA, epithelial proteins such as p63 and K18 were detected in RA-treated hESCs. Interestingly, N2-supplemented medium showed greater differentiation than unconditioned hESC medium, presumably because of an absence of Knockout Serum Replacer, which can induce TGF-β/Activin/Nodal signaling. Subculture of differentiated colonies in keratinocyte medium yielded hESC-derived K14⁺populations having a higher purity when compared to EB-derived K14+ cells. After five weeks of differentiation, 87% of hESC-derived K14⁺ populations typically expressed K14.

Essentially pure populations of K14⁺ cells were obtained when hESC-derived p63⁺ cells were sub-cultured onto gelatin or Collagen IV substrates, comparable to primary keratinocytes. Flow cytometry analysis of K14 expression measured 90% K14⁺ cells in the primary cultures; whereas K14 expression in the hESC-derived K14+ cells was 96%. The K14⁺ cells exhibited a lower expansion potential than primary cultures (only 10-15 population doublings upon subculture), though the proliferative capacity of subcultures decreased with the amount of time spent in adherent culture prior to passaging. In addition, colony forming K14+ cells were obtained at a frequency of at least 0.003 from differentiated hESCs plated, which is a six-fold increase over previously reported methods in immunocompromised mice. See Iuchi et al., supra.

BMP-4 synergized with RA in direct differentiation of hESCs to K14⁺ cells. hESCs were directly differentiated in N2 medium containing RA, BMP-4 or Noggin (a BMP inhibitor). After differentiation for six days, RNA was analyzed for expression of epithelial and neuroectodermal transcripts. ΔNp63 was elevated in all RA-treated cell samples, regardless of status of BMP signaling. In addition, neuroepithelial gene expression (e.g., Nestin, Pax6 and Sox1) was downregulated in RA-treated cell samples; however, BMP-4 inhibition mitigated this effect. In contrast, RA and BMP-4 synergistically induced p63 expression.

Subsequent culture of differentiated hESCs in keratinocyte medium further defined the roles of RA and BMP. K14⁺ yield was only slightly affected in RA+/−cultures exposed to BMP-4, whereas yield decreased when BMP signaling was inhibited by Noggin. Similar results were observed in RA-treated EBs, although the level of induction was lower compared to direct differentiation of hESCs. In addition, hESC-derived K14-positive cells generated via exposure to RA expressed terminal differentiation markers in high-density and Ca²⁺ containing cultures. Small areas of stratification appeared in extended cultures with or without Ca²⁺; the top layers expressed involucrin, filaggrin and in some cases an epidermal-specific keratin, K10. These markers are characteristic of keratinocytes. Moreover, some remaining cells express K18; others expressed K3/K12, which are characteristic of corneal epithelial cell markers. Confluent cell cultures in Ca²⁺-containing, defined keratinocyte medium detached from the substrate as an intact epithelial sheet using Dispase®, and contracted accordingly. RA-induced hESCs produced relatively pure cultures that expressed terminal differentiation markers and formed coherent epithelial sheets under appropriate conditions. This high-efficiency method coupled with the self-renewal capacity of undifferentiated hESCs provides a means of generating large quantities of non-transformed keratinocytes.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

The invention claimed is:

1. A method of generating p63-positive cells, the method comprising the step of:
culturing embryoid bodies in a medium comprising a retinoid and optionally a bone morphogenetic protein for about two days to about nine days, each in an amount sufficient to generate p63-positive cells.

2. The method of claim 1, wherein the embryoid bodies are human embryoid bodies.

3. The method of claim 1, wherein the retinoid is selected from the group consisting of tretinoin, alitretinoin and isotretinoin.

4. The method of claim 1, wherein the bone morphogenetic protein is bone morphogenetic protein-4.

5. The method of claim 1, wherein the retinoid is provided at a concentration between about 0.1 μM to about 10 μM.

6. The method of claim 1, wherein the bone morphogenetic protein is provided at a concentration between about 5 ng/ml to about 50 ng/ml.

7. The method of claim 1, wherein the cells are also K18 positive.

8. The method of claim 1, wherein the medium is a defined medium.

9. A method of generating p63 positive cells, the method comprising the step of:
culturing pluripotent cells in a medium comprising a retinoid and optionally a bone morphogenetic protein for about five days to about seven days, each in an amount sufficient to generate p63 positive cells.

10. The method of claim 9, wherein the pluripotent cells are selected from the group consisting of human embryonic stem cells and induced pluripotent stem cells.

11. The method of claim 9, wherein the retinoid is selected from the group consisting of tretinoin, alitretinoin and isotretinoin.

12. The method of claim 9, wherein the bone morphogenetic protein is bone morphogenetic protein-4.

13. The method of claim 9, wherein the retinoid is at a concentration between about 0.1 μM to about 10 μM.

14. The method of claim 9, wherein the bone morphogenetic protein is at a concentration between about 5 ng/ml to about 50 ng/ml.

15. The method of claim 9, wherein the cells are also K18 positive.

16. The method of claim 9, wherein the medium is a defined medium.

17. A method of generating K14-positive cells, the method comprising the step of:
culturing p63-positive cells to confluence in a defined serum-free medium to obtain K14-positive cells, wherein the cells are on an adherent surface, and wherein the medium does not comprise a retinoid and bone morphogenetic protein.

18. The method of claim 17, wherein the adherent surface comprises a material selected from the group consisting of collagen, fibronectin, gelatin, glycosaminoglycans, laminin, Matrigel®, osteocalcin, osteonectin and combinations thereof.

19. A method of generating filaggrin- and involucrin-positive cells, the method comprising the step of:
culturing K14-positive cells to confluence in a defined serum-free medium to obtain filaggrin- and involucrin-positive cells, wherein the cells are on an adherent surface.

20. The method of claim 19, wherein the adherent surface comprises a material selected from the group consisting of collagen, fibronectin, gelatin, glycosaminoglycans, laminin, Matrigel®, osteocalcin, osteonectin and combinations thereof.

21. The method of claim 19, wherein the cells are also K10 positive.

22. A method of generating K3/K12-positive cells, the method comprising the step of:
culturing K14-positive cells to confluence in a defined serum-free medium to obtain K3/K12-positive cells, wherein the cells are on an adherent surface.

23. The method of claim 22, wherein the adherent surface comprises a material selected from the group consisting of collagen, fibronectin, gelatin, glycosaminoglycans, laminin, Matrigel®, osteocalcin, osteonectin and combinations thereof.

24. A population of cultured K14-positive cells, wherein at least about 85% of the cells express K14.

25. The population of cultured cells of claim 24, wherein the K14-positive cells are produced from cells selected from the group consisting of embryoid bodies and pluripotent cells.

26. A population of cultured filaggrin- and involucrin-positive cells, wherein at least about 85% of the cells express filaggrin and involucrin.

27. The population of cultured cells of claim 26, wherein the cells also express K10.


SEQUENCE LISTING (20 sequences)

SEQ ID NO: 1 - DNA, 20 bp, Artificial Sequence
  Synthetic oligonucleotide
caccgtcaaggctgagaacg

SEQ ID NO: 2 - DNA, 20 bp, Artificial Sequence
  Synthetic oligonucleotide
gccccacttgattttggagg

SEQ ID NO: 3 - DNA, 18 bp, Artificial Sequence
  Synthetic oligonucleotide
gggagcggtgaagatgga

SEQ ID NO: 4 - DNA, 22 bp, Artificial Sequence
  Synthetic oligonucleotide
tcatgttgctcacggaggagta

SEQ ID NO: 5 - DNA, 20 bp, Artificial Sequence
  Synthetic oligonucleotide
tgcttccctgagacccagtt

SEQ ID NO: 6 - DNA, 25 bp, Artificial Sequence
  Synthetic oligonucleotide
gatcacttctttcctttgcatcaag

SEQ ID NO: 7 - DNA, 20 bp, Artificial Sequence
  Synthetic oligonucleotide
tgaagggcaatcacaacagg

SEQ ID NO: 8 - DNA, 20 bp, Artificial Sequence
  Synthetic oligonucleotide
tgaccccaacatgacctctg

SEQ ID NO: 9 - DNA, 20 bp, Artificial Sequence
  Synthetic oligonucleotide
caatgcggggaggagaagtc

SEQ ID NO: 10 - DNA, 20 bp, Artificial Sequence
  Synthetic oligonucleotide
ctctggaccaaactgtggcg

SEQ ID NO: 11 - DNA, 20 bp, Artificial Sequence
  Synthetic oligonucleotide
ggcaggtattacgagactgg

SEQ ID NO: 12 - DNA, 20 bp, Artificial Sequence
  Synthetic oligonucleotide
cctcatctgaatcttctccg

SEQ ID NO: 13 - DNA, 20 bp, Artificial Sequence
  Synthetic oligonucleotide
ccgtcttgctgctgatgact

SEQ ID NO: 14 - DNA, 22 bp, Artificial Sequence
  Synthetic oligonucleotide
ggccttttacttcctcttcgtg

SEQ ID NO: 15 - DNA, 20 bp, Artificial Sequence
  Synthetic oligonucleotide
aagatggtgcgacaaacaag

SEQ ID NO: 16 - DNA, 20 bp, Artificial Sequence
  Synthetic oligonucleotide
agagagcatcgaaggtggag

SEQ ID NO: 17 - DNA, 20 bp, Artificial Sequence
  Synthetic oligonucleotide
ggaaaacaatgcccagactc

SEQ ID NO: 18 - DNA, 20 bp, Artificial Sequence
  Synthetic oligonucleotide
gtggaatacgtccaggtggc

SEQ ID NO: 19 - DNA, 20 bp, Artificial Sequence
  Synthetic oligonucleotide
gaccattgaggacctgagga

SEQ ID NO: 20 - DNA, 20 bp, Artificial Sequence
  Synthetic oligonucleotide
attgatgtcggcttccacac